(19) United States
(12) Patent Application Publication
(10) Pub. No.: US 2007/0243385 A1
(43) Pub. Date: Oct. 18, 2007

(54) Water Resistant Undercast Padding

(75) Inventors: John C. Evans, Lancashire (GB); Shitij Chabba, Charlotte, NC (US)

Correspondence Address: ADAMS EVANS P.A., Suite 2350 Charlotte Plaza, 201 South College Street, CHARLOTTE, NC, 28244, US

(73) Assignee: BSN MEDICAL, INC., Charlotte, NC (US)

(21) Appl. No.: 11/571,124

(22) PCT Filed: Feb. 18, 2005
(86) PCT No.: PCT/US05/05256

§ 371 Date: Jun. 27, 2007

Related U.S. Application Data
(60) Provisional application No. 60/582,053, filed on Jun. 22, 2004.

(51) Int. Cl. A61F 5/00

(52) U.S. Cl. 428/409

(57) ABSTRACT

An undercast padding for being placed on an injured limb before application of a cast (10). The padding includes an elongate foam substrate layer (11) having first and second major sides and first and second opposed side edges, first and second moisturebreathable film layers (12, 13) applied to the first and second major sides, the first and second opposed side edges remaining uncovered by the first and second films, and an array of ventilation holes (16, 15) formed in spaced-apart relation to each other through the foam substrate layer and first and second film layers. The array of ventilation holes (16, 15) preferably is formed of first and second rows of longitudinally spaced-apart holes extending along the length of the substrate layer in closely spaced-apart relation to the respective first and second opposed side edges.

### TECHNICAL FIELD AND BACKGROUND OF THE INVENTION

This invention relates to a relates to an undercast padding of the type used to protect the skin of a patient from the relatively rigid material of a cast, such as constructed of plaster of Paris or synthetic cast tape.

Traditional cast padding is made from cotton or synthetic fibers and offers poor or no water resistant capability. A cast is typically worn for a period of 6-8 weeks. During this period of time, traditional casts having water absorbent stockinette often promote skin maceration, discomfort and breed bacteria-causing odor as perspiration and water from washing and bathing migrates to and through the undercast padding. The undercast padding remains wet or damp for an extended period of time, causing the problems mentioned above.

The present invention provides a more conformable, waterproof padding at a reduced cost as compared to products already available in the market. The present invention is directed to a construction that overcomes the drawbacks of waterproof undercast padding such as that found in U.S. Pat. Nos. 5,102,711 and 5,277,954. For example, the film/foam substrate has higher elongation and provides a higher stretch during application that results in a better conforming padding, which can be easily molded around a limb. Due to the improved padding/cushioning as compared to similar products known in the prior art, the undercast padding of the present invention requires less layers during application. The present invention also has an adhesive layer on either or both film surfaces. The tacky surface, when applied away from the skin, adheres to itself to form a smoother underlayer of a cast. Additionally, it provides a non-slip effect under the cast tape.

From an economic perspective, water resistant undercast padding can help alleviate skin maceration problems which generally require additional treatment or therapy and eliminates the need for frequent cast changes. The present invention accommodates bathing, showering and contact with water without significant penetration of water onto the skin. In addition, the padding of the present invention is believed to provide improved conformability, cushioning, breathability, ease of application and a low profile as compared to products currently in the market.

### SUMMARY OF THE INVENTION

Therefore, it is an object of the invention to provide a water resistant undercast padding.

It is another object of the invention to provide a undercast padding that is comfortable when worn under a plaster or synthetic cast tape, splint, brace or any other orthopedic device.

It is another object of the invention to provide a undercast padding that is relatively thin and thus provides a low profile undercast padding layer even when properly overlapped during application.

It is another object of the invention to provide a undercast padding that is breathable.

These and other objects of the present invention are achieved in the preferred embodiments disclosed below by providing an undercast padding product, comprising an elongate foam substrate layer having first and second major sides and first and second opposed side edges, first and second moisture-breathable film layers applied to the first and second major sides, the first and second opposed side edges remaining uncovered by the first and second films, and an array of ventilation holes formed in spaced-apart relation to each other through the foam substrate layer and first and second film layers.

According to one preferred embodiment of the invention, the array of ventilation holes comprises first and second rows of longitudinally spaced-apart holes extending along the length of the substrate layer in closely spaced-apart relation to the respective first and second opposed side edges.

According to another preferred embodiment of the invention, the substrate layer comprises a foam selected from the group consisting of acrylics, nitrites, polyurethane, styrene-butadiene rubber, ethylene vinyl alcohol (EVA), polyvinyl acetate (PVAC), neoprene, polyvinylidene chloride (PVDC), polyvinyl chloride (PVC), polyolefins such as polyethylene (PE) or combinations and blends thereof with silicones and other suitable polymeric materials.

According to yet another preferred embodiment of the invention, the film layers comprise a film selected from the group consisting of low density polyethylene (LDPE), high density polyetheylene (HDPE), polyethylene terephthalate (PET), polyurethane (PU), polytetrafluoroethylene (PTFE), expanded polytetrafluoroethylene (e-PTFE), and polyvinylidene chloride (PVDC).

According to yet another preferred embodiment of the invention, at least some of the holes in the array of ventilation holes comprises a first, relatively large hole formed in only the substrate layer and a second, relatively small hole formed in the first and second film layers in registration within the first, relatively large hole.

According to yet another preferred embodiment of the invention, the first and second film layers are adhered to the substrate by a pressure sensitive adhesive or by heat and pressure only.

According to yet another preferred embodiment of the invention, the undercast padding includes an embossed pattern applied to at least one of the first and second major surfaces thereof.

According to yet another preferred embodiment of the invention, the undercast padding includes an embossed pattern applied to the first and second major surfaces thereof.

According to yet another preferred embodiment of the invention, first and second stabilizer grooves are embossed along the length thereof between the first and second rows of longitudinally spaced-apart holes and respective first and second side edges of the substrate. The padding is also embossed with a series of width-wise stabilizer grooves which can be evenly or unevenly distributed along the length of the padding.

According to yet another preferred embodiment of the invention, the undercast padding product comprises an elongate foam substrate layer having first and second major sides and first and second opposed side edges, wherein the substrate layer comprises a foam selected from the group consisting of acrylics, nitrites, polyurethane, styrene-butadiene rubber, ethylene vinyl alcohol (EVA), polyvinyl acetate (PVAC), neoprene, polyvinylidene chloride (PVDC), polyvinyl chloride (PVC), polyolefins such as polyethylene (PE) or combinations and blends thereof. First and second moisture-breathable film layers are applied to the first and second major sides. The first and second opposed side edges remain uncovered by the first and second films. The film layers comprise a film selected from the group consisting of low density polyethylene (LDPE), high density polyetheylene (HDPE), polyethylene terephthalate (PET), polyurethane (PU), polytetrafluoroethylene (PTFE), expanded polytetrafluoroethylene (e-PTFE), and polyvinylidene chloride (PVDC). An array of ventilation holes is formed in spaced-apart relation to each other through the foam substrate layer and first and second film layers, wherein the array of ventilation holes comprises first and second rows of longitudinally spaced-apart holes extending along the length of the substrate layer in closely spaced-apart relation to the respective first and second opposed side edges. The first and the second film layer may be constructed using the same or different material. The array of ventilation hopes can either be longitudinally aligned or randomly scattered along the length of the padding.

### BRIEF DESCRIPTION OF THE DRAWINGS

Some of the objects of the invention have been set forth above. Other objects and advantages of the invention will appear as the invention proceeds when taken in conjunction with the following drawings, in which:

FIG. 1 is a photograph of one surface of the undercast padding according to one embodiment of the invention;

FIG. 2 is an exploded perspective view of the undercast padding during assembly according to one embodiment of the invention;

FIG. 3 is a perspective view of the undercast padding according to one embodiment of the invention;

FIG. 4 is an environmental view of a roll of the undercast padding being applied to the wrist and forearm of a patient;

FIGS. 5-9 are sequential views showing application of a synthetic cast tape to a forearm protected with the undercast padding according to an embodiment of the invention.

FIGS. 10-16 are views of an undercast padding having various alternative hole patterns.

### DESCRIPTION OF THE PREFERRED EMBODIMENT AND BEST MODE

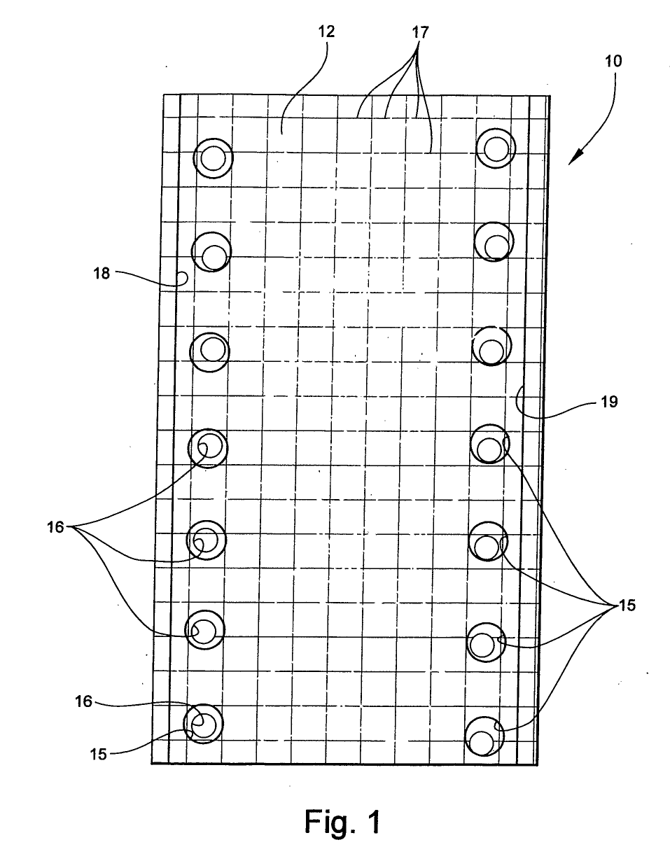

Referring now specifically to the drawings, a undercast padding according to the present invention is illustrated in FIG. 1 and shown generally at reference numeral 10. While the undercast padding 10 can be formed in any desired width or length, the undercast padding 10 shown in FIG. 1 is 7.5 cm (3 in.) wide.

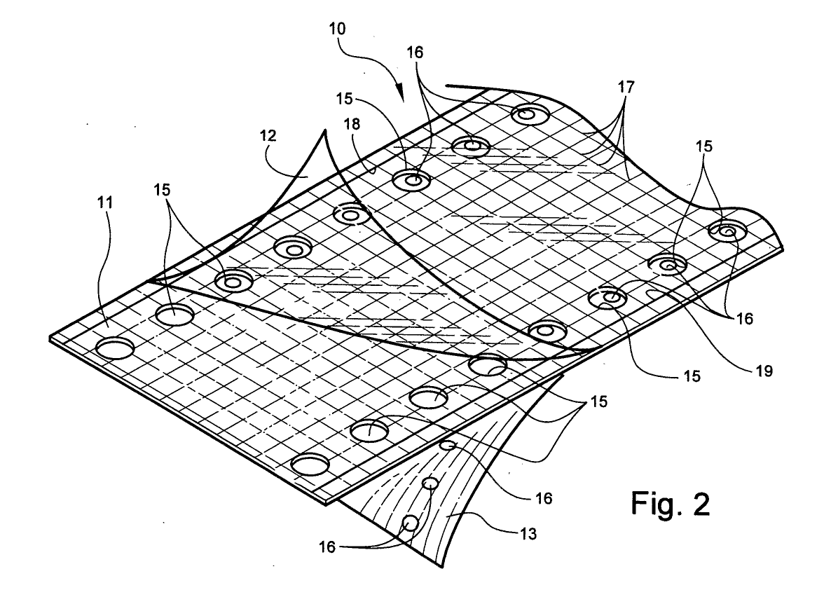

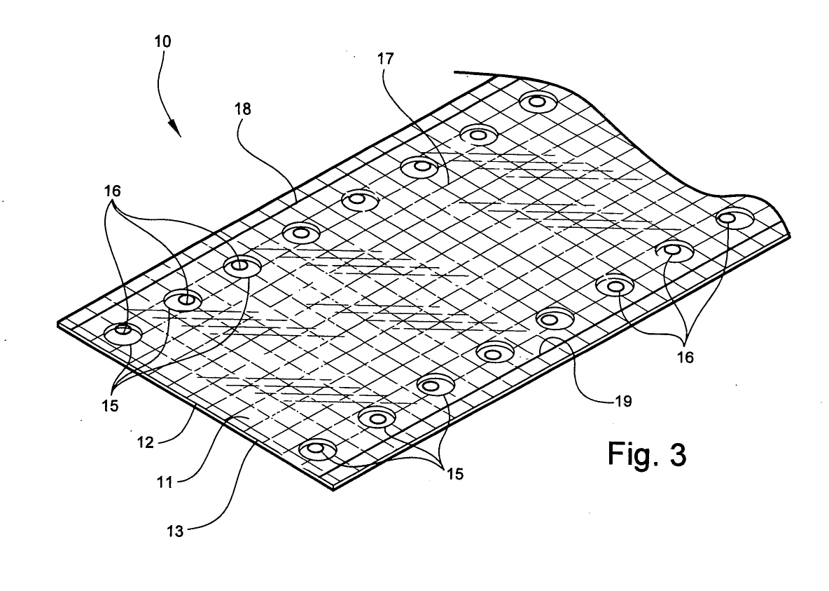

Referring now to FIGS. 2 and 3, the undercast padding 10 comprises a foam substrate 11 overlaid with two breathable film layers 12 and 13. The film layers 12 and 13 can be attached to the foam substrate 11 by thermal or ultrasonic adherence, pressure-sensitive adhesive, heat and pressure, or a combination of these methods. The foam substrate 11 is punched to form rows of longitudinally spaced-apart ventilation holes 15 extending in laterally spaced-apart relation to the opposing side edges of the substrate 11. The holes 15 are formed before application of the film layers 12 and 13.

Application of the film layers 12 and 13 is achieved by moving a strip of the substrate 11 along a process line where strips of film with a pressure sensitive adhesive thereto are dispensed from rolls and applied to opposite sides of the substrate 11 and then adhered by passing the assembly through a pair of nip rolls. The assembled undercast padding 10 is then passed through another set of rolls that emboss a shallow grid pattern 17, of for example, squares, and a pair of elongate stabilizer grooves 18 and 19 to both surfaces of the undercast padding 10. The stabilizer grooves 18 and 19 are principally to reduce the thickness of the foam substrate 11 along this line and thus reduce water penetration while permitting passage of air. The grooves 18 and 19 also stabilize the film layers 12 and 13 and maintain them in adhered relation to the cut edges of the substrate 11.

The assembled undercast padding 10 is then passed through a second hole-punching operation where smaller ventilation holes 16, see FIG. 3, are punched in registration with the larger holes 15, so that small plugs of film are removed from the film layers 12 and 13 only in the area overlying the holes 15. The edges of the film layers 12 and 13 defining these holes 16 may be sealed together, or may be completely or partially unsealed to provide additional ventilation.

The assembled undercast padding 10 is then rolled into a suitable-length roll and packaged for shipment and storage until ready for use.

The foam utilized for the substrate 11 can be formed from acrylics, nitriles, polyurethane, styrene-butadiene rubber, ethylene vinyl alcohol (EVA), polyvinyl acetate (PVAC), neoprene, polyvinylidene chloride (PVDC), polyvinyl chloride (PVC), polyolefins such as polyethylene (PE) or combinations and blends thereof. The foam for the substrate 11 can be manufactured either as an open cell, closed cell or reticulated cell with a rigid, semi-rigid or flexible hardness. The substrate 11 can have a pore density in the range of 5-150 ppi with an elongation value of 25%-700%. The substrate 11 in the preferred embodiment is a flexible polyester polyurethane-based reticulated, fully open pore, designated P-100Z from Illbruck, Inc. having the following values:

| Density | 1.90 | |
|---|---|---|
| IFD @25% | 0.40 | psi |
| @65% | 0.65 | psi |
| Tensile Strength | 35 | psi |
| Elongation | 400% | |
| Tear Strength | 3.9 | lb/in. |

| | |
|---|---|
| 50% Compression Set | 12 |
| Pore Size (ppi) | 100 |
| Reticulation Method | Thermal |

The film layers 12 and 13 can be constructed from the same material or can be of two different types. The film layers 12 and 13 should offer a breathable yet moisture-impervious flexible film layer, and can be constructed using low density polyethylene (LDPE), high density polyetheylene (HDPE), polyethylene terephthalate (PET), polyurethane (PU), polytetrafluoroethylene (PTFE), expanded polytetrafluoroethylene (e-PTFE), polyvinylidene chloride (PVDC) or any suitable polymer offering the desired properties. The film layers 12 and 13 prevent or significantly reduce water or perspiration from coming in direct contact with the foam, thus giving it a significant degree of water-resistance. The surface of the film layers 12 and/or 13 can be etched, patterned, dimpled or flat. The film layers 12 and 13 can be attached to the foam substrate 11 by thermal, ultrasonic, pressure-sensitive adhesive, heat and pressure, or a combination of these. Film thickness can vary from 0.01 mm to 5 mm. The film layers should have a moisture vapor transmission rate of at least 500 g/m²/24 hr. The preferred film layers 12 and 13 are a 2 mil polyurethane-based material flexible, breathable, moisture impervious and fungal-resistant film. This polyurethane film has a moisture vapor transmission rate of 100 g/m²/24 hr as per ASTM E-96 test method.

The adhesive used can be any pressure sensitive adhesive approved for use in medical products that come into contact with the skin. The adhesive layer can be 0.02-2.5 mil thick.

The grooves 18 and 19 can be at a distance of 1 mm-5 mm away from the respective opposing edges of the undercast padding 10. In the preferred embodiment, the grooves 18 and 19 are at a distance of 3 mm from the opposed outer edges of the undercast padding 10. The holes 15 along the edges of the undercast padding 10 provide additional stretch to the material as well as prevent water from being trapped inside the padding once the cast tape has been applied.

The holes 15 also improve the moisture vapor transmission rate for the padding, leading to improved comfort for the wearer. For example, where application is required to include enclosure of an elbow, water can remain trapped inside the padding in materials. The holes 15 can be circular, elliptical, square, rectangular or any contour, and can be spaced equally or unequally apart. In the preferred embodiment, the holes 15 are circular, 100 mm away from the edges on either side and have a diameter of 8 mm. The holes 16 in the film layers 12 and 13 in the area overlying the holes 15 are 4 mm in diameter.

The substrate of the present invention is coated with a 1.6 mil thick pressure sensitive acrylic adhesive on one surface of the film to allow for bonding of the material around the limb during application.

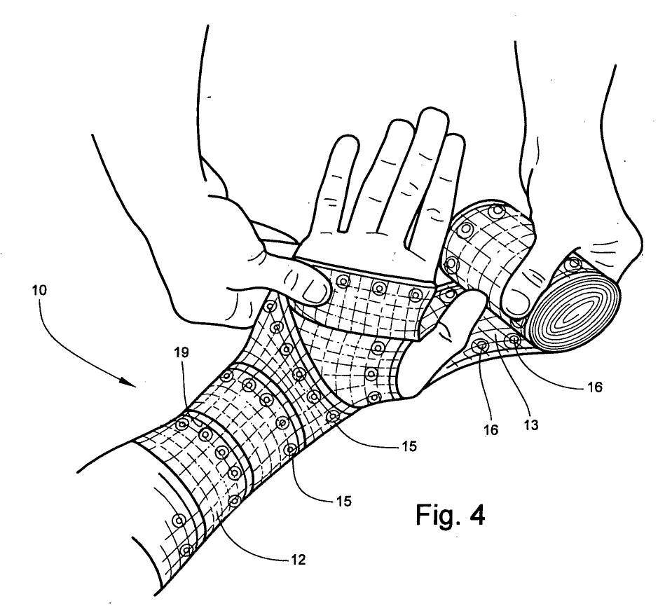

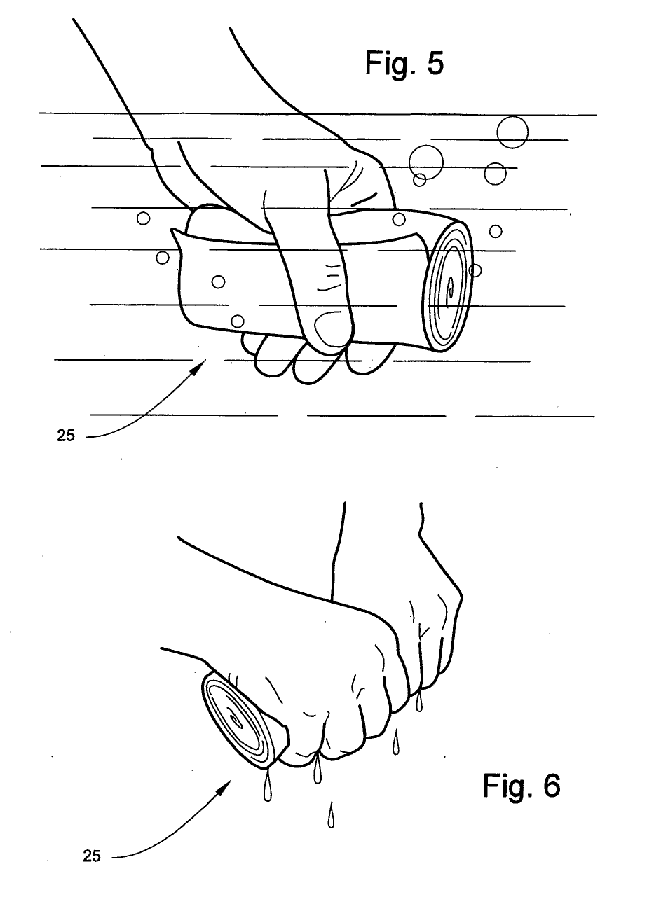

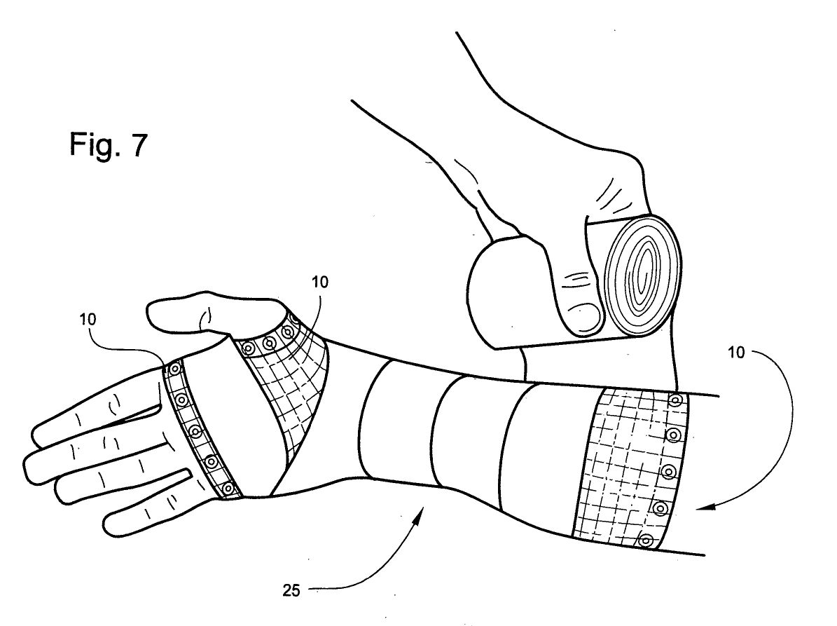

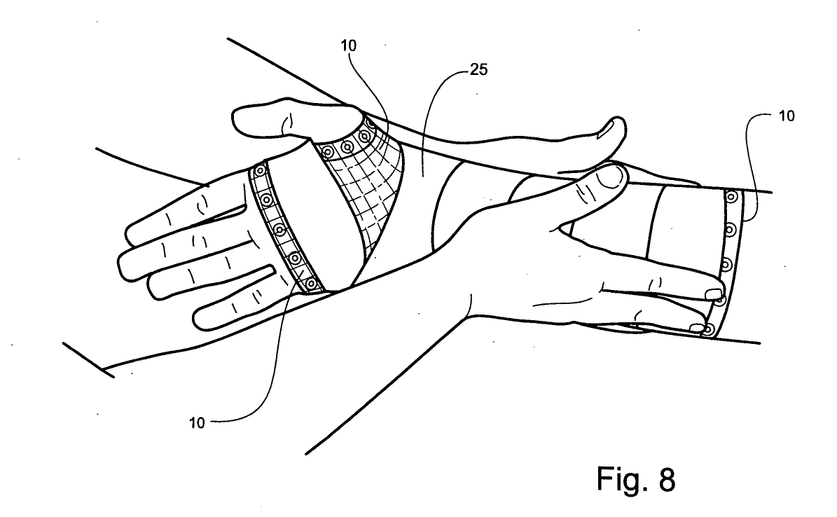

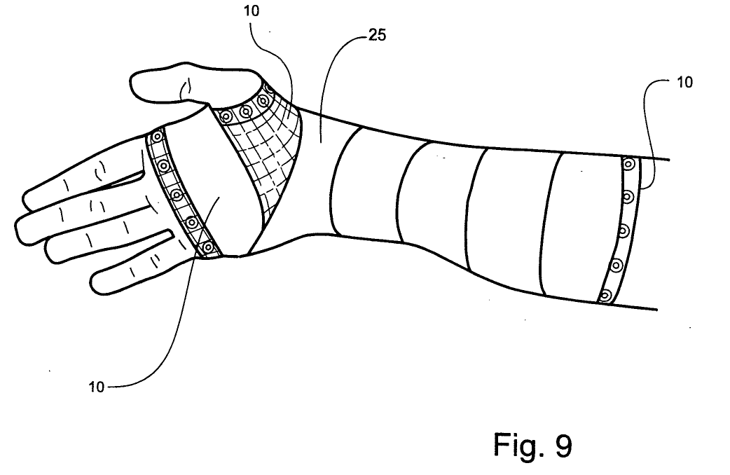

Referring now to FIG. 4, the undercast padding is applied to the injured limb in a conventional manner. As is shown in FIGS. 5-9, after application of the undercast padding, a conventional cast tape 25 is wetted, FIG. 5, excess water removed by wringing, FIG. 6, and applied to the injured limb, FIGS. 7-9, taking care in the usual manner to avoid overlapping the undercast padding 10 on opposite ends, leaving a small length of exposed undercast padding 10.

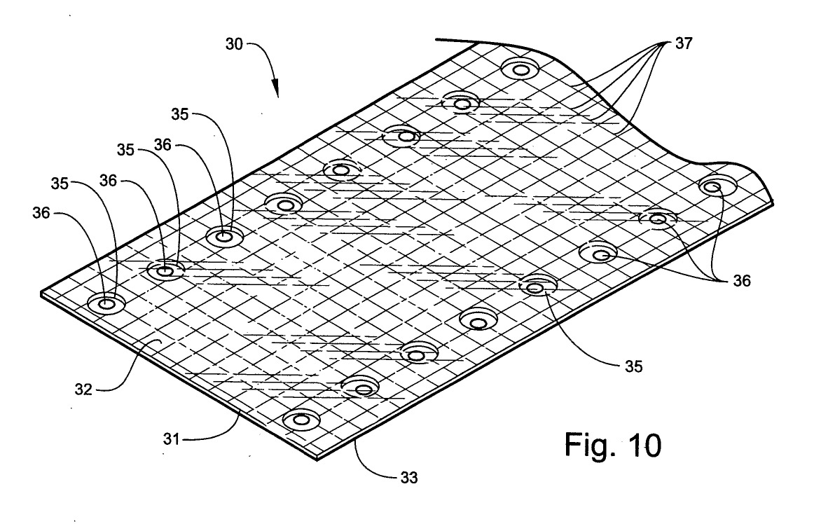

Referring now to FIGS. 10-16, several variations in the arrangement of the holes are illustrated. Specifically, in FIG. 10, a undercast padding 30 is shown, comprising a comprises a foam substrate 31 overlaid with two breathable film layers 32 and 33. The film layers 22 and 33 are attached to the foam substrate 31, as described above with reference to undercast padding 10, by thermal or ultrasonic adherence, or by a pressure-sensitive adhesive, or a combination of these methods. The foam substrate 31 is punched to form rows of longitudinally spaced-apart ventilation holes 35 extending in laterally spaced-apart relation to the opposing side edges of the substrate 31. The holes 35 are formed before application of the film layers 32 and 33.

In the particular design shown in FIG. 10, the stabilizer grooves are omitted, and the adherence of the film layers 32 and 33 are relied upon to maintain structural integrity and resistance against water intrusion.

The assembled undercast padding 30 is then passed through a second hole-punching operation where smaller ventilation holes 36 are punched in registration with the larger holes 35, so that small plugs of film are removed from the film layers 32 and 33 only in the area overlying the holes 35.

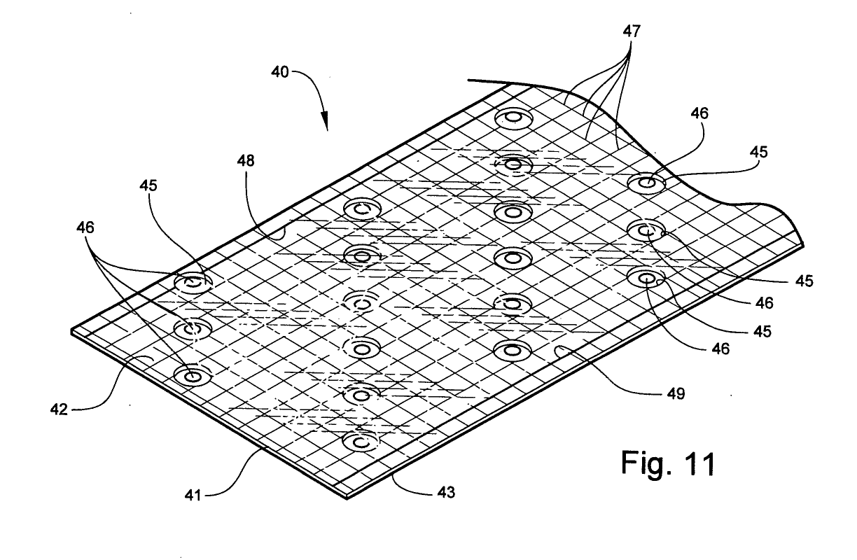

Referring now to FIG. 11, a undercast padding 40 is shown, comprising a comprises a foam substrate 41 overlaid with two breathable film layers 42 and 43. The film layers 42 and 43 are attached to the foam substrate 41, as described above. The foam substrate 41 is punched to form rows of spaced-apart ventilation holes 45 extending in diagonally spaced-apart rows.

Application of the film layers 42 and 43 is achieved as described above. The assembled undercast padding 40 is then passed through another set of rolls that emboss a shallow grid pattern 47, of for example, squares. Stabilizer grooves 48, 49 are optionally provided, as described above.

The assembled undercast padding 40 is then passed through a second hole-punching operation where diagonally spaced-apart smaller ventilation holes 46 are punched in registration with the larger holes 45, so that small plugs of film are removed from the film layers 42 and 43 only in the area overlying the holes 45.

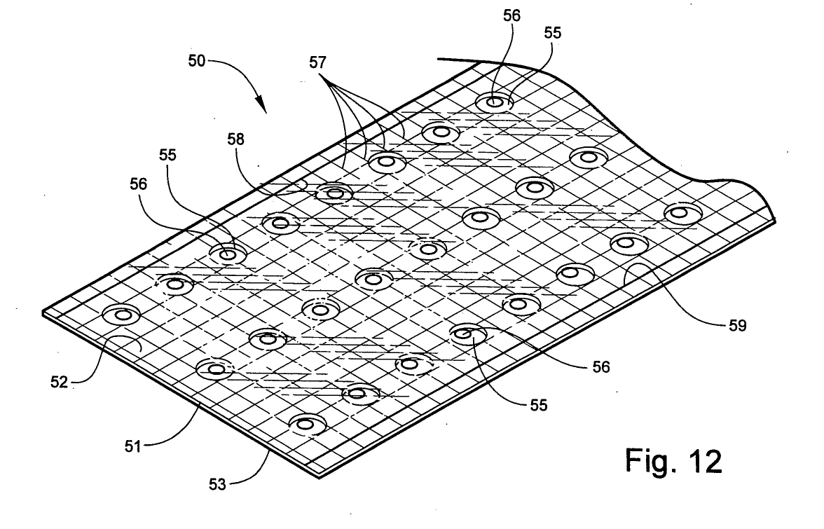

Referring now to FIG. 12, a undercast padding 50 is shown, comprising a comprises a foam substrate 51 overlaid with two breathable film layers 52 and 53. The film layers 52 and 53 are attached to the foam substrate 51, as described above. The foam substrate 51 is punched to form three rows of spaced-apart ventilation holes 55 extending along the length of the substrate 51.

Application of the film layers 52 and 53 is achieved as described above. The assembled undercast padding 50 is then passed through another set of rolls that emboss a shallow grid pattern 57, of for example, squares. Stabilizer grooves 58, 59 are optionally provided, as described above.

The assembled undercast padding 50 is then passed through a second hole-punching operation where smaller ventilation holes 56 are punched in registration with the larger holes 55, so that small plugs of film are removed from the film layers 52 and 53 only in the area overlying the holes 55.

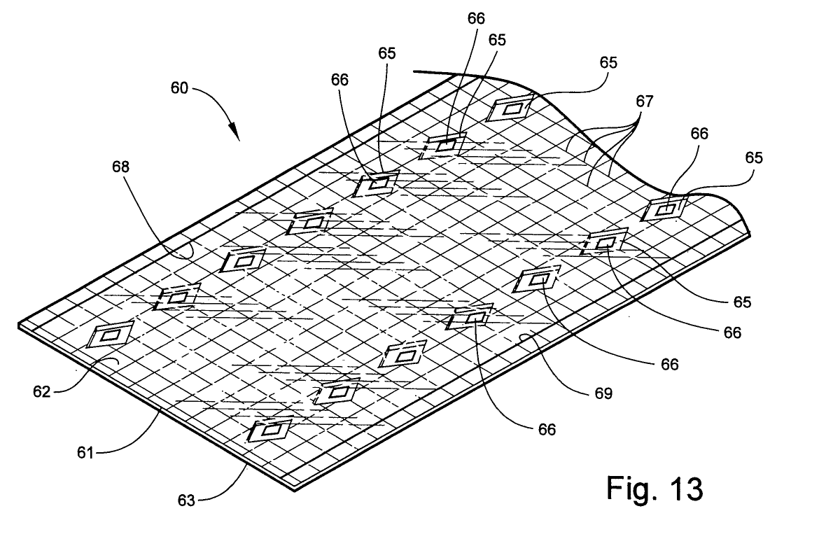

Referring now to FIG. 13, a undercast padding 60 is shown, comprising a comprises a foam substrate 61 overlaid with two breathable film layers 62 and 63. The film layers 62 and 63 are attached to the foam substrate 61, as described above. The foam substrate 61 is punched to form two rows of spaced-apart, diamond-shaped ventilation holes 65 extending along the length of the substrate 61.

Application of the film layers 62 and 63 is achieved as described above. The assembled undercast padding 60 is then passed through another set of rolls that emboss a shallow grid pattern 67, of for example, squares. Stabilizer grooves 68, 69 are optionally provided, as described above.

The assembled undercast padding 60 is then passed through a second hole-punching operation where smaller ventilation holes 66 are punched in registration with the larger holes 55, so that small plugs of film are removed from the film layers 62 and 63 only in the area overlying the holes 65. The holes 66 may be diamond-shaped, as shown, or may be an entirely different shape.

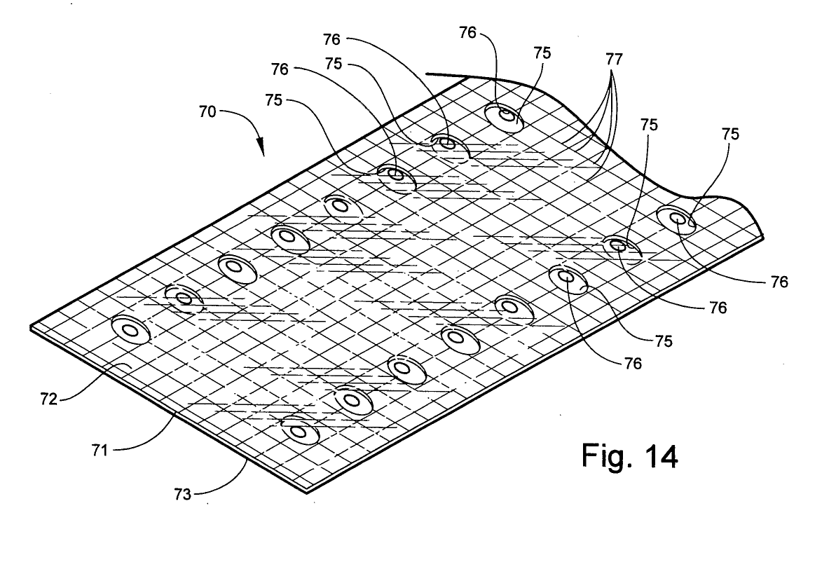

Referring now to FIG. 14, a undercast padding 70 is shown, comprising a comprises a foam substrate 71 overlaid with two breathable film layers 72 and 73. The film layers 72 and 73 are attached to the foam substrate 71, as described above. The foam substrate 71 is punched to form two rows of spaced-apart oval ventilation holes 75 extending along the length of the substrate 71. As shown, the oval shape of the holes 75 may provide a greater degree of stretch across the width of the undercast padding 70 than along its length. Reorienting the long axis of the oval holes 75 may provide a greater degree of stretch along the length of the undercast padding 70 than across its width.

Application of the film layers 72 and 73 is achieved as described above. The assembled undercast padding 70 is then passed through another set of rolls that emboss a shallow grid pattern 77, of for example, squares. Stabilizer grooves 78, 79 are optionally provided, as described above.

The assembled undercast padding 70 is then passed through a second hole-punching operation where smaller ventilation holes 76 are punched in registration with the larger holes 75, so that small plugs of film are removed from the film layers 72 and 73 only in the area overlying the holes 75. The ventilation holes 76 may be oval in shape, as are the larger ventilation holes 75, or may be an entirely different shape.

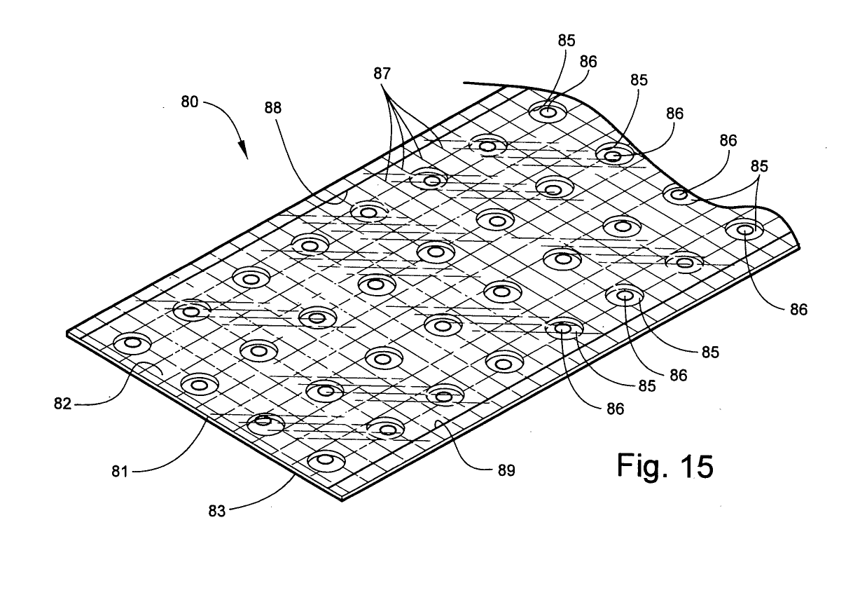

Referring now to FIG. 15, a undercast padding 80 is shown, comprising a comprises a foam substrate 81 overlaid with two breathable film layers 82 and 83. The film layers 82 and 83 are attached to the foam substrate 81, as described above. The foam substrate 81 is punched to form four rows of spaced-apart round ventilation holes 85 extending along the length of the substrate 81. As shown, the holes 85, due to the regular spacing, may also be considered to extend diagonally along the length of the substrate 81.

Application of the film layers 82 and 83 is achieved as described above. The assembled undercast padding 80 is then passed through another set of rolls that emboss a shallow grid pattern 87, of for example, squares. Stabilizer grooves 88, 89 are optionally provided, as described above.

The assembled undercast padding 80 is then passed through a second hole-punching operation where smaller ventilation holes 86 are punched in registration with the larger holes 85, so that small plugs of film are removed from the film layers 82 and 83 only in the area overlying the holes 85. The ventilation holes 86 may be the same shape as are the larger ventilation holes 85, or may be an entirely different shape.

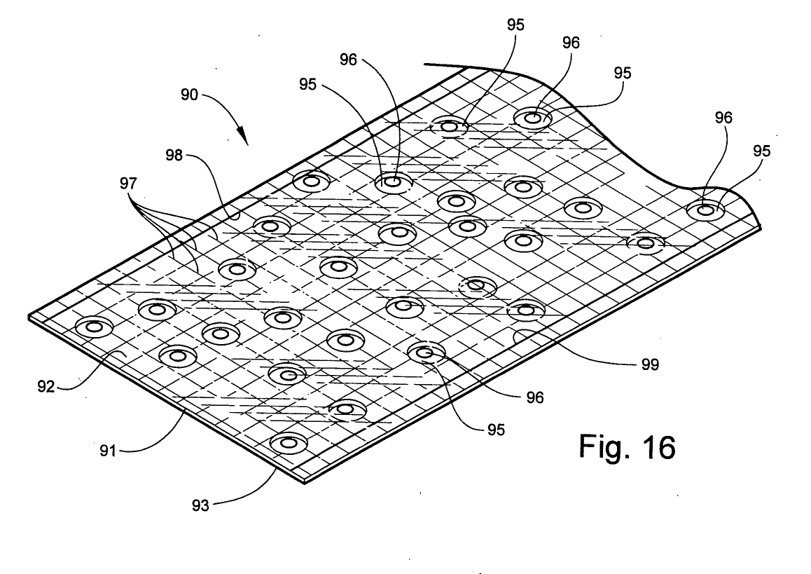

Referring now to FIG. 16, a undercast padding 90 is shown, comprising a comprises a foam substrate 91 overlaid with two breathable film layers 92 and 93. The film layers 92 and 93 are attached to the foam substrate 91, as described above. The foam substrate 91 is punched with randomly-appearing, spaced-apart ventilation holes 95 extending along the length of the substrate 91.

Application of the film layers 92 and 93 is achieved as described above. The assembled undercast padding 90 is then passed through another set of rolls that emboss a shallow grid pattern 97, of for example, squares. Stabilizer grooves 98, 99 are optionally provided, as described above.

The assembled undercast padding 90 is then passed through a second hole-punching operation where smaller, randomly-appearing, spaced ventilation holes 96 are punched in registration with the larger holes 95, so that small plugs of film are removed from the film layers 92 and 93 only in the area overlying the holes 95. The ventilation holes 96 may be the same shape as are the larger ventilation holes 95, or may be an entirely different shape. Of course, the holes 95 and 96 only appear to be randomly spaced. A pattern presenting a random appearance permits the holes 95 and 96 to be punched in registration with each other.

An undercast padding is described above. Various details of the invention may be changed without departing from its scope. Furthermore, the foregoing description of the preferred embodiment of the invention and the best mode for practicing the invention are provided for the purpose of illustration only and not for the purpose of limitation—the invention being defined by the claims.

We claim:

1. An undercast padding product, comprising:
(a) an elongate foam substrate layer having first and second major sides and first and second opposed side edges;
(b) first and second moisture-breathable film layers applied to the first and second major sides, the first and second opposed side edges remaining uncovered by the first and second films; and
(c) an array of ventilation holes formed in spaced-apart relation to each other through the foam substrate layer and first and second film layers.

2. An undercast padding product according to claim 1, wherein the array of ventilation holes comprises first and second rows of longitudinally spaced-apart holes extending along the length of the substrate layer in closely spaced-apart relation to the respective first and second opposed side edges.

3. An undercast padding product according to claim 1, wherein the substrate layer comprises a foam selected from the group consisting of acrylics, nitriles, polyurethane, styrene-butadiene rubber, ethylene vinyl alcohol (EVA), polyvinyl acetate (PVAC), neoprene, polyvinylidene chloride (PVDC), polyvinyl chloride (PVC), polyolefins such as polyethyelene (PE) or combinations and blends thereof.

4. An undercast padding product according to claim 1, wherein the film layers comprise a film selected from the group consisting of low density polyethylene (LDPE), high density polyetheylene (HDPE), polyethylene terephthalate (PET), polyurethane (PU), polytetrafluoroethylene (PTFE), expanded polytetrafluoroethylene (e-PTFE), and polyvinylidene chloride (PVDC).

5. An undercast padding product according to claim 1, wherein at least some of the holes in the array of ventilation holes comprises a first, relatively large hole formed in only the substrate layer and a second, relatively small hole formed in the first and second film layers in registration within the first, relatively large hole.

6. An undercast padding product according to claim 1, wherein the first and second film layers are adhered to the substrate.

7. An undercast padding product according to claim 1, and including an embossed pattern applied to at least one of the first and second major surfaces thereof.

8. An undercast padding product according to claim 1, and including an embossed pattern applied to the first and second major surfaces thereof.

9. An undercast padding product according to claim 2, and including first and second stabilizer grooves embossed along the length thereof between the first and second rows of longitudinally spaced-apart holes and respective first and second side edges of the substrate.

10. An undercast padding product, comprising:
(a) an elongate foam substrate layer having first and second major sides and first and second opposed side edges, wherein the substrate layer comprises a foam selected from the group consisting of acrylics, nitrites, polyurethane, styrene-butadiene rubber, ethylene vinyl alcohol (EVA), polyvinyl acetate (PVAC), neoprene, polyvinylidene chloride (PVDC), polyvinyl chloride (PVC), polyolefins such as polyethyelene (PE) or combinations and blends thereof;
(b) first and second moisture-breathable film layers applied to the first and second major sides, the first and second opposed side edges remaining uncovered by the first and second films, wherein the film layers comprise a film selected from the group consisting of low density polyethylene (LDPE), high density polyetheylene (HDPE), polyethylene terephthalate (PET), polyurethane (PU), polytetrafluoroethylene (PTFE), expanded polytetrafluoroethylene (e-PTFE), and polyvinylidene chloride (PVDC); and
(c) an array of ventilation holes formed in spaced-apart relation to each other through the foam substrate layer and first and second film layers, wherein the array of ventilation holes comprises first and second rows of longitudinally spaced-apart holes extending along the length of the substrate layer in closely spaced-apart relation to the respective first and second opposed side edges.

11. An undercast padding product according to claim 10, wherein at least some of the holes in the array of ventilation holes comprises a first, relatively large hole formed in only the substrate layer and a second, relatively small hole formed in the first and second film layers in registration within the first, relatively large hole.

12. An undercast padding product according to claim 10, wherein the first and second film layers are adhered to the substrate by a pressure sensitive adhesive.

13. An undercast padding product according to claim 10, and including an embossed pattern applied to at least one of the first and second major surfaces thereof.

14. An undercast padding product according to claim 10, and including an embossed pattern applied to the first and second major surfaces thereof.

15. An undercast padding product according to claim 11, and including first and second stabilizer grooves embossed along the length thereof between the first and second rows of longitudinally spaced-apart holes and respective first and second side edges of the substrate.
